(19) United States
(12) Patent Application Publication
(10) Pub. No.: US 2007/0037281 A1
(43) Pub. Date: Feb. 15, 2007

(54) Method for differentiating stem cells in cells that produce a pancreatic hormone

(75) Inventor: Charli Kruse, Herrnburg (DE)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: FRAUNHOFER-GESELLSCHAFT ZUR FORDERUNG DER ANGEWANDTEN FORSCHUNG E.V., Munchen (DE)

(21) Appl. No.: 10/561,628

(22) PCT Filed: Jun. 23, 2004
(86) PCT No.: PCT/EP04/06799

§ 371 Date: Feb. 9, 2006

(30) Foreign Application Priority Data
Jun. 23, 2003 (DE) 103 28 280.7
Apr. 8, 2004 (DE) 10 2004 017 473.3

(51) Int. Cl. C12N 5/08; C12N 15/09

(52) U.S. Cl. 435/366; 435/455

(57) ABSTRACT

Described is a process for generating cells producing pancreatic hormone by cultivating and differentiating stem cells obtained from differentiated exocrine glandular tissue of an organism. The use of the cells producing pancreatic hormone is also described.

The present invention relates to processes for the differentiation of stem cells into cells that produce a pancreatic hormone, especially into insulin-producing cells, cells producing pancreatic hormone, cellular compositions that contain cells producing pancreatic hormone and especially are generated with the said process, applications of such cells and cellular compositions, especially pharmaceutical applications of these cells and cellular compositions.

The differentiation of stem cells into insulin or other pancreatic hormones producing cells is known (see DE 102 90 025 T1, WO 02/059278). The traditional differentiation techniques have significant disadvantages as far as the precursor cells for the cell differentiation as well as the complex process conditions that must be observed for cell differentiation are concerned. The traditional processes are based in practice on the differentiation of the cells from aggregates of embryonic stem cells (so-called embryoids). However, the massive usability of embroids for cell differentiation is excluded for a number of reasons and especially ethical considerations. DE 102 90 025 T1 does suggest the differentiation of adult stem cells. However, the adult stem cells obtained with the traditional processes yield the desired differentiation results only to a limited extent.

The object of the invention is to provide improved processes for the differentiation of stem cells into cells that produce at least one pancreatic hormone, with which the limitations concerning the use of embryonic stem cells are avoided and that are nevertheless suitable for a massive use under simple process conditions. The object of the invention consists in particular in the achievement of an improved process for the generation of cells producing pancreatic hormone for transplantation in patients suffering from pancreatic diseases, especially diabetes.

This object is achieved by a process having the features of Claim 1, a cell having the features of Claim 22 and a cell composition having the features of Claim 24. Advantageous embodiments and applications are derived from the dependent claims.

As concerns the process, the above-mentioned object is achieved by cultivating and differentiating non-embryonic stem cells that were extracted from the tissue of a differentiated exocrine gland of an organism. A particular advantage of this process consists in the generation of cells that produce pancreatic hormone (in the following: hormone-producing cells) without the previously used differentiation from embryonic stem cells. The inventors observed that the adult stem cells isolated from exocrine glandular tissue are pluripotent and show a high ability to divide and a strong growth. This provides an effective source for cells capable of differentiation from which the hormone-producing cells can be generated in a large scale.

The exocrine glandular tissue used in accordance with the invention can stem from an adult organism, a juvenile organism or non-human fetal organism, preferably a postnatal organism. The concept “adult” as it is used in the present application therefore refers to the development stage of the initial tissue and not to that of the donor organism from which the tissue stems. “Adult” stem cells are non-embryonic stem cells.

The exocrine glandular tissue is preferably isolated from a salivary gland, lachrymal gland, sebaceous gland, sudoriferous gland, from glands of the genital tract including the prostate, or from gastrointestinal tissue, including the pancreas, or from secretory tissue of the liver. In a strongly preferred embodiment, acinar tissue is concerned. It is very especially preferred that the acinar tissue stems from the pancreas, the parotid gland or the submandibular gland.

A further important advantage of the process in accordance with the invention resides in the fact that the stem cells can be effectively obtained from living donor organisms, e.g., from salivary glands of mammals or from human salivary glands without the donor organism being substantially adversely affected. This is especially advantageous for ethical viewpoints as well as regards the possibility of further observation of the donor organism regarding any diseases.

According to a first embodiment of the invention the stem cells isolated primarily from the organism are used as source for the further cultivation and differentiation into hormone-producing cells. This variant has the advantage that it is especially simple to conduct the process. The desired differentiated cells can be gained directly from a primary culture. Alternatively, it is envisaged according to a modified embodiment of the invention that at first an aggregation of the stem cells isolated from the organism to so-called organoid bodies occurs. This variant has the advantage that an effective reservoir for greater amounts of differentiated cells is created with the organoid bodies. The inventors observed that the stem cells isolated from the exocrine glandular tissue form organoid bodies that show a strong growth to tissue bodies with diameters up to a few millimeters or more when supplied with nutrients.

According to a first variant of the use of organoid bodies as source for the desired hormone-producing cells the invention provides that the differentiation of the stem cells occurs in the organoid bodies and that the differentiated hormone-producing cells are subsequently removed from the organoid bodies. According to a second variant stem cells are isolated secondarily from the organoid bodies in a non-differentiated state, cultivated and differentiated into the desired hormone-producing cells. This embodiment has the advantage that it is particularly simple to conduct the process since the organoid bodies show a spontaneous emigration and growth of stem cells or differentiated cells in adhesion culture that are then available for further cultivation and differentiation.

The process in accordance with the invention can basically be carried out in that hormone-producing cells that formed spontaneously from the primarily or secondarily isolated stem cells or from the organoid bodies are selected and propagated further. According to a preferred embodiment of the invention a stimulation of the cell culture is envisaged in the differentiation of the hormone-producing cells. The stimulation has the advantage of an increased effectiveness and rate of the generation of the desired hormone-producing cells. According to a first variant, after the differentiation of the stem cells into the hormone-producing cells, their stimulated propagation in a cultivation medium is envisaged. According to a second variant the stimulation to a stimulated differentiation of the stem cells into the desired hormone-producing cells is envisaged.

According to the invention the stimulation may comprise one or more of the following stimulation treatments that may be carried out simultaneously or successively. A treatment may be envisaged with supernatants of a primary culture of the endocrinal pancreas or of cell lines of the endocrinal pancreas, a co-cultivation with differentiated cells of the endocrinal pancreas or with cell lines derived from them, a treatment (imprinting) with immobilized or dissolved molecular differentiation factors provided in the liquid phase or a gene activation in the stem cell. Furthermore, stimulation can be achieved by the addition of already differentiated, hormone-producing cells and/or other substances, for instance, hormones, or cell types that influence the differentiation.

If the imprinting is performed with immobilized growth factors, differentiation factors are preferably used that are fixed on a movable carrier that can be positioned relative to the stem cells. This can advantageously achieve a purposeful differentiation of individual stem cells or of certain stem cell groups. The carrier is, for instance, a synthetic substrate that has advantages for a purposeful design with the differentiation factors, or a biological cell on whose cellular membrane the differentiation factors are arranged.

If, according to a further preferred embodiment of the invention, an identification and selection of the differentiated cells from the cell culture is envisaged, this may yield advantages for the further use of the pancreatic hormone producing cells generated. In particular, a cellular composition can be provided that consists entirely or for the most part of hormone-producing cells. If the selection is performed with known sorting methods such as, for instance, with a preparative cell sorting method or a sorting in a fluidic microsystem, this may result in advantages for the compatibility with traditional cellular biological procedures.

A further advantage of the identification and selection resides in the fact that cells that are not identified as hormone-producing cells and are correspondingly not selected from the processed culture can be subjected to a further cultivation and differentiation. This can advantageously increase the yield of the process in accordance with the invention.

According to preferred further variants of the invention stem cells are obtained from tissue from secretory glands or glands of the gastrointestinal tract of the organism for generating the hormone-producing cells. The stem cells are isolated in particular from tissue that consists of acinar tissue or contains acinar tissue. The obtention from the pancreas may yield advantages for the use of further tissue parts of the pancreas for the said stimulation. The obtention from the salivary gland may yield advantages for the protective treatment of the donor organism.

Preferred donor organisms are vertebrates, especially mammals such as, for instance, a human being. When using human stem cells the isolation of the stem cells is performed from non-embryonic states, that is, from differentiated tissue in the juvenile phase or the adult phase. In the case of non-human donor organisms it is basically also possible to use differentiated tissue in the fetal state.

The hormone-producing cells produced in accordance with the invention are preferably used therapeutically. According to a first variant a human treatment with hormone-producing cells obtained from animal organisms can be envisaged. Furthermore, treatments of a human being with hormone-producing cells obtained from another human being are possible. The autologous treatment of a human with hormone-producing cells obtained from his own juvenile or adult stem cells is especially preferred. The treatment can relate to pancreatic diseases, metabolic syndromes or metabolic diseases, especially diabetes, hyperglycemia or an impaired glucose tolerance. Insulin is produced with particular preference as the pancreatic hormone.

An independent subject matter of the invention is an isolated cell producing pancreatic hormone that was differentiated from a stem cell stemming from differentiated endocrine glandular tissue of an organism. Such an isolated cell is preferably obtained with the process in accordance with the invention.

A further independent subject matter of the invention is a cellular composition containing a plurality of such cells producing pancreatic hormone. The cellular composition is preferably obtained by the process in accordance with the invention. According to a preferred embodiment of the invention the cellular composition may contain other cells or materials that form, for instance, a matrix. Accordingly, a further subject matter of the invention is an artificial islet of Langerhans.

According to a preferred embodiment of the invention the cellular composition comprises a casing or a 3-dimensional matrix in which the hormone-producing cells and other cell types are arranged. The casing or 3-dimensional matrix consists, for instance, of alginate, collagen, implantable materials, polymers (biopolymers or synthetic polymers). A semi-permeable material that permits the hormone (for instance, insulin) but not the cells to pass to the outside is used. The other cell types comprise, for instance, stem cells and/or neighboring cells of islets of Langerhans in the pancreatic tissue. The formation of the casing (capsule) is preferred since in particular the hormone-producing cells are immobilized especially effectively with it and prevented, if necessary, from further growth. The capsule-cell composite or matrix-cell composite described here has a diameter of a few 100 μm to a few mm.

A further independent subject matter of the invention is a pharmaceutical composition with at least one hormone-producing cell or cellular composition produced in accordance with the invention and a pharmaceutical carrier substance. Any material that is known for this function in the art of pharmacy may be used as pharmaceutical, in particular liquid carrier substance.

Other advantages and details of the invention are described in the following with reference made to the attached drawings.

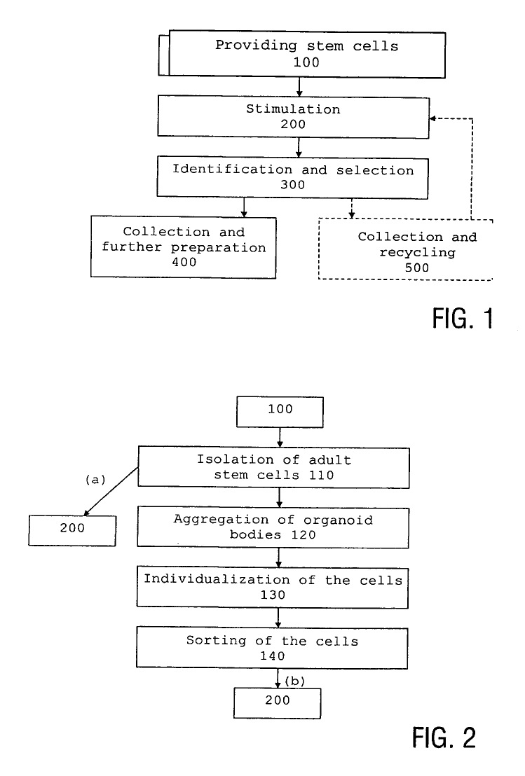

FIG. 1 shows a flowchart of a differentiation process in accordance with an embodiment of the invention.

FIG. 2 shows a flowchart illustrating various variants of providing stem cells.

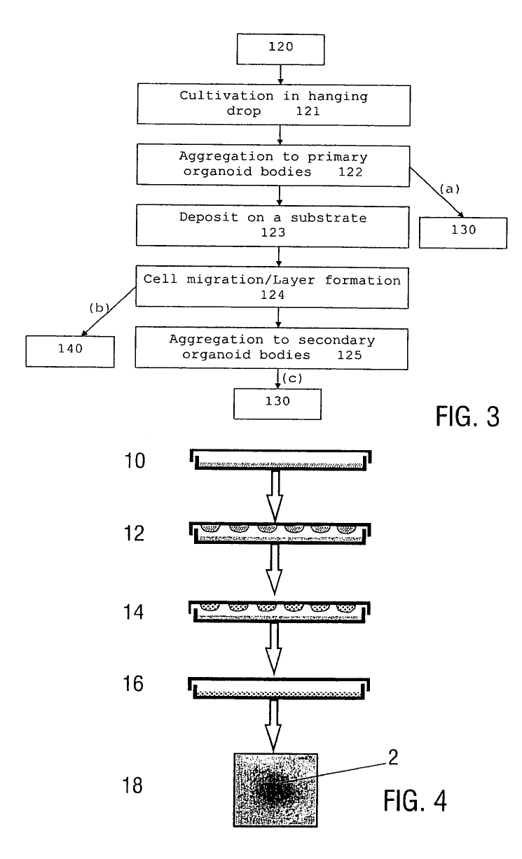

FIG. 3 shows a flowchart illustrating various variants of the aggregation of stem cells to organoid bodies.

FIG. 4 shows a procedure for producing organoid bodies.

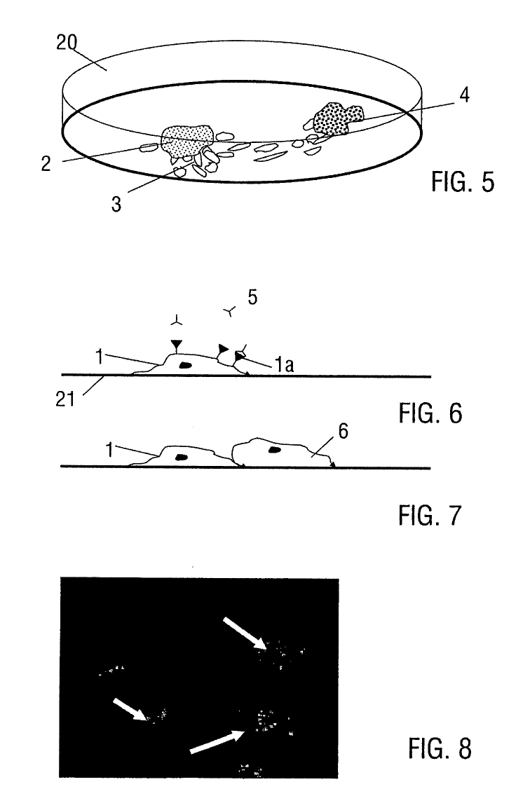

FIG. 5 shows an illustration of the cultivation of organoid bodies.

FIGS. 6 and 7 show schematic illustrations of the imprinting of stem cells by molecular signal factors, and

FIG. 8 shows an illustration of the demonstration of the insulin production of cells differentiated in accordance with the invention.

### (I) ISOLATION AND AGGREGATION OF STEM CELLS FROM A DIFFERENTIATED EXOCRINE GLAND OF A LIVING ORGANISM TO ORGANOID BODIES

The cultivation and differentiation in accordance with the invention of stem cells obtained from differentiated exocrine glandular tissue of an organism comprise the steps illustrated in FIG. 1. At first, the isolation of stem cells from the organism (step 100) is performed in order to provide a source for the stimulation and differentiation into the hormone-producing cells. Details of the isolation and various variants of sources for the further process steps are explained below. The stimulation (step 200) of a cell culture used as source is subsequently performed within the framework of the differentiation. After the differentiation, an identification of the hormone-producing cells and a corresponding selection is performed in step 300. The selected hormone-producing cells are collected and further prepared, if necessary, for, e.g., a pharmaceutical application (step 400). Furthermore, a recycling of the non-differentiated cells into the initial culture in order to be re-exposed to the stimulation and differentiation may be envisaged (step 500).

Different variants of providing non-differentiated stem cells for the further stimulation are shown in FIGS. 2 and 3. According to FIG. 2 the isolation of stem cells from the donor organism (step 110, see below for examples) is performed at first. Starting from these primarily isolated stem cells, a direct transition to the stimulation (step 200) can be made according to a first variant (a). Alternatively, an aggregation of the stem cells to organoid bodies is performed at first in step 120. Details of the aggregation step are compiled in FIGS. 3 and 4. Stem cells or already differentiated cells can be individualized and/or sorted (step 140) from the organoid bodies in step 130, during which according to variant (b) the further stimulation of the differentiation or the propagation of the already differentiated cells can be continued.

According to FIG. 3 the aggregation of stem cells to organoid bodies according to step 120 comprises the following partial steps. At first, the cultivation of the stem cells in suspended drops (step 121, see below for example) is performed. Alternatively, the cultivation can be performed in an agitated culture or in a suspended state without contact with solid boundary surfaces in electromagnetic field cages. The aggregation to primary organoid bodies is performed in the suspended drop or the corresponding three-dimensional suspension culture (step 122). These bodies can be used as source for the individualization of cells. According to variant (a) in FIG. 4 the jump to step 130 in FIG. 2 takes place here. Alternatively, the primary organoid bodies are first deposited on a substrate in a cultivation medium for an adhesion culture (step 123). A cell migration and the layer formation take place on the substrate (step 124, see FIG. 5). As a result, after step 124, individualized stem cells or differentiated cells are present so that according to variant (b) in FIG. 3 the jump to step 140 in FIG. 2 can immediately take place.

A particularity of the organoid bodies in adhesion culture consists in the formation of secondary organoid bodies from the cell layer formed in the adhesion culture (step 125). Cells can be individualized again from the secondary organoid bodies or correspondingly derived organoid bodies according to variant (c) in FIG. 3 (jump to step 130 in FIG. 2).

The individualization of the differentiated cells from an adhesion culture takes place according to known processes, for instance, using marker substances characteristic for the pancreatic hormones. For example, a trypsination of the cells differentiated in adhesion culture and their transfer into a suspension are performed. An identification is performed in this suspension, for instance, using a known cell sorting equipment with a fluidic microsystem, with a preparative cell sorter process or with magnetic beads to which antibodies are coupled that only couple with the hormone-producing cells in the suspension. Surface markers are purposefully sought and the hormone-producing cells selected with specific surface markers in the preparative cell sorter process.

The identification of the cells sought can also be performed in the cell culture, for instance, on the basis of morphological distinguishing features by which the hormone-producing cells are distinguished from the undifferentiated cells or other cell types. Morphological distinguishing features refer, for instance, to the geometry of the cell or the arrangement of the cell nucleus or of granular components in the cell.

Furthermore, the identification and selection can be performed by way of a cellular electrophoretic analysis or an analysis of another specific, native property such as, for instance, the surface mobility of the hormone-producing cells.

Furthermore, multistage identifications and selections or identifications and selections combined from the said techniques can be envisaged.

According to the scheme shown in FIG. 4, in order to obtain the cells, acinar tissue, preferably from a salivary gland or the pancreas, is taken in culture in mechanically and enzymatically comminuted form (step 10 in FIG. 2). Contrary to the indications of Bachem et al., Gastroenterol. 115:421-432 (1998), and Grosfils et al., Res. Comm. Chem. Pathol. Pharmacol. 79:99-115 (1993), no tissue blocks are cultivated from which cells are supposed to grow but rather the tissue is more strongly comminuted, with the proviso that the cell aggregates of the acini remain intact to the greatest extent possible.

These cells and cell aggregates are cultivated in culture vessels for several weeks. The medium is changed every 2 to 3 days, and all differentiated cells are removed at this time. The cells persisting in culture are non-differentiated cells with unlimited capability of division.

Similar cells have been isolated and described under the same conditions from the pancreas and designated as a type of myofibroblasts or pancreatic star cells (Bachem et al., 1998). However, in contrast to the cells of the present invention, an unlimited capability of division could not be observed. Furthermore, these cells could also not be passaged in an unlimited manner without losing vitality.

In a second step (12) approximately 400 to 800 cells each are cultivated in 20 μl medium in suspended drops. To this end the drops are put on the cover of bacteriological Petri dishes, turned over and placed over the Petri dish filled with medium so that the drops hang downward.

As a result of this type of cultivation the cell aggregates (14) designated as organoid bodies form within 48 hours that are transformed for approximately 6 days into a suspension culture (16). The partial view (18) in FIG. 4 shows a microscopic view of such an organoid body 2.

The formation of the above-mentioned secondary organoid bodies is illustrated in FIG. 5. At first, the primary organoid bodies 2 form a monolayer on the substrate of the adhesion culture such as, for instance, on the bottom of culture dish 20 by a migration or growth of cells 3 from which monolayer the secondary organoid bodies 4 then grow out. A further multiplication of the cellular material is generated with the cultivation of primary organoid bodies 2 to secondary organoid bodies 4. The hormone-producing cells can be obtained from each of primary or secondary organoid bodies 2, 4.

Further Exemplary Embodiments of the Isolation and Aggregation of Stem Cells

The isolation and aggregation of stem cells is explained in detail in the following non-limiting examples.

The general working instructions that are customary for processes for the cultivation of biological cells and in particular of mammalian cells are to be observed. A sterile environment, in which the process is to be carried out, even if no further description for this is provided, is to be maintained in every instance. The following buffers and media were used:

| | |
|---|---|
| HEPES stock solution | 2.3 83 g HEPES per 100 ml A. bidest. |
| (pH 7.6) | |
| HEPES eagle medium | 90 ml modified eagle medium (MEM) |
| (pH 7.4) | 10 ml HEPES stock solution |
| Isolation medium | 32 ml HEPES eagle medium |
| (pH 7.4) | 8 ml 5% BSA in bidest. water |
| | 300 μl 0.1 M CaCl₂ |
| | 100 ml Trasylol (200,000 KIE) |
| Digestion medium | 20 ml isolation medium |
| (pH 7.4) | 4 ml collagenase |
| | (collagenase NB 8 from Serva) |
| Incubation medium | Dulbecco's modified eagle medium (DMEM) |
| Culture medium | Dulbecco's modified eagle medium (DMEM) |
| | DMEM + 4500 mg/l glucose + L-glutamine − |
| | pyruvate + 20% FCS (inactivated) + 1 ml/100 ml |
| | penicillin/streptomycin (10000 E/10000 μg/ml) |
| | or |
| | DMEM + 10% autologous plasma + |
| | 1 ml/100 ml penicillin/streptomycin. |
| | heat to 37° C. before use |
| Differentiation | 380 ml DMEM |
| medium (I) | 95 ml FCS inactivated for 30 min at 54° C. |
| | 5 ml glutamine (GIBCO BRL) |
| | 5 ml (3.5 μl β-mercaptoethanol to 5 ml PBS) |
| | 5 ml nonessential amino acids (GIBCO BRL) |
| | 5 ml penicillin/streptomycin (GIBCO BRL) |
| | (10000 E/10000 (μg/ml) |

Instead of fetal calf serum (FCS) in the culture medium and differentiation medium, autologous plasma or, less preferably, autoserum of the tissue donor may also be used, if necessary. This is significant in particular when the tissue donor is identical to the subsequent recipient of the stem cells or differentiated cells derived therefrom. Such an autologous treatment is preferred in order to prevent a possible rejection reaction.

The culture medium may also contain, instead of the DMEM medium, another suitable basic medium known for the cultivation of eukaryotic cells, in particular mammalian cells, in which medium the differentiated cells die off and the desired stem cells propagate. The isolation medium, incubation medium and differentiation medium may also contain another customary and suitable basic medium.

Further differentiation media that may be used for the stimulation provided in accordance with the invention consist in the supernatants of primary cultures or of derived cell lines of the endocrine pancreas or in cell suspensions with differentiated cells or derived cells of the endocrine pancreas. In particular, cells or cell groups that are located in the endocrine tissue of the pancreas in the vicinity of the islets of Langerhans can be used.

The following examples 1 and 2 describe in detail two working protocols for the isolation and cultivation of adult pluripotent stem cells from acinar pancreatic tissue. Examples 1 and 2 refer to the isolation of stem cells from rats. The isolation from other mammals, for instance, pigs, is performed by analogy. Example 3 describes a corresponding protocol for the isolation from acinar tissue of the salivary gland.

### EXAMPLE 1

1. Preparation of the Tissue and Isolation of the Cells

10 ml digestion medium are injected slowly and without air bubbles into the Ductus pancreaticus of 2 to 3 years old rats with a syringe and a blunt cannula in the rat. The entire pancreas is thereby inflated and can therefore be removed and prepared more easily. The pancreas is then transferred into a glass beaker and another 5 ml digestion medium is added. After the fatty tissue and lymph nodes have been removed, the tissue is very finely comminuted in the glass beaker with fine scissors, fatty tissue floating on the top removed by suction and the suspension is subsequently gassed for 1 min with Carbogen (repeating if necessary) and incubated for 20 min at 37° C., covered with aluminum foil, in an agitator at 200 cycles/min. The medium is then carefully removed by suction, the tissue comminuted again with scissors and the pieces of tissue are washed twice with 10 ml isolation medium each and 5 ml digestion medium are again added to the tissue.

After gassing with carbogen again for approximately 1 min and incubation for 15 minutes at 370 in an agitator at 200 cycles/min the pieces of tissue are comminuted by being successively drawn up into a 10 ml, 5 ml, 2 ml and 1 ml glass pipette and pressed through a single-layer filter fabric. The cells individualized in this manner are now washed five times in incubation medium (37° C.), gassed with Carbogen and centrifuged 5 min each time at 90 g. The pellet obtained finally is re-suspended in incubation medium, gassed and distributed onto tissue culture dishes.

2. Cultivation of the Cells

The tissue culture dishes with the isolated cells are cultivated in an incubator at 37° C. and 5% CO₂. The medium is changed every 2 to 3 days, and all differentiated cells are removed at this time.

On the seventh day in culture, the cells are passaged with a solution consisting of 2 ml PBS, 1 ml trypsin and 2 ml incubation medium. In the course of this, the cells separate from the bottom of the culture dish. The cell suspension is centrifuged for 5 minutes, the supernatant is removed by suction and the cells are re-suspended in 2 ml incubation medium, transferred to a medium-sized cell culture flask and 10 ml incubation medium are added. The media is changed every three days.

On the fourteenth day in culture, the cells are passaged again, but this time with 6 ml PBS, 3 ml trypsin and 6 ml incubation medium. The cell suspension is centrifuged for 5 minutes, the supernatant is removed by suction and the cells are re-suspended in 6 ml incubation medium, transferred to 3 medium cell culture flasks and 10 ml incubation medium are added to each flask.

The cells are cultivated further and passaged and sown until the cells attain a semi-confluent to confluent state.

### EXAMPLE 2

Pancreatic acini were obtained from male Sprague-Dawley rats (20 to 300 g) that had been narcotized (CO₂) and exsanguinated via the dorsal aorta. A cannula was introduced transduodenally into the pancreatic duct and 10 ml digestion medium containing HEPES eagle medium (pH 7.4), 0.1 mM HEPES buffer (pH 7.6), 70% (vol./vol.) modified eagle medium, 0.5% (vol./vol.) Trasylol (Bayer AG, Leverkusen, Germany), 1% (wt./vol.) bovine serum albumin), 2.4 mM CaCl₂ and collagenase (0.63 P/mg, Serva, Heidelberg, Germany) was injected into the pancreas from behind.

Prior to the removal, the pancreas was partially freed of adhering fatty tissue, lymph nodes and blood vessels. Then, healthy pancreatic tissue was collected in digestion medium (at 20° C., lesser metabolism), the pancreatic tissue very finely comminuted with scissors, fatty tissue floating on the top was removed by suction and the tissue suspension was gassed with Carbogen (Messer, Krefeld, Germany) without the jet passing into the medium with the cells (reduction of mechanical stress) and adjusted therewith to pH 7.4. The suspension was then incubated in a 25 ml Erlenmeyer flask (covered with aluminum foil) under constant agitation (150-200 cycles per minute) at 37° C. in 10 ml digestion medium. After 15 to 20 minutes, the fat floating on top and the media were removed by suction and the tissue was comminuted again and rinsed with medium without collagenase (repeating the procedure at least twice, preferably until cell fraction is transparent), whereupon digestion medium was added and the mixture gassed again for approximately 1 minute with Carbogen. A digestion with collagenase followed again for 15 minutes at 37° C. in an agitator using the same buffer. After the digestion, the acini were dissociated by being successively drawn up and ejected through 10 ml, 5 ml and 2 ml glass pipettes with narrow openings and filtered by a single-layer nylon mesh (Polymon PES-200/45, Angst & Pfister AG, Zürich, Switzerland) with a mesh size of approximately 250 μm. The acini were centrifuged (at 37° C. and 600-800 rpm in a Beckman GPR centrifuge, corresponding to approximately 90 g) and cleaned further by washing in incubation medium containing 24.5 mM HEPES (pH 7.5), 96 mM NaCl, 6 mM KCl, 1 mM MgCl₂, 2.5 mM NaH₂PO₄, 0 mM CaCl₂, 11.5 mM glucose, 5 mM sodium pyruvate, 5 mM sodium glutamate, 5 mM sodium fumarate, 1% (vol./vol.) modified eagle medium, 1% (wt./vol.), bovine serum albumin, equilibrated with Carbogen and adjusted to pH 7.4. The washing procedure (centrifugation, removal by suction, re-suspension) was repeated five times. Unless otherwise indicated, the work is performed at approximately 20° C. for the above isolation.

The acini were re-suspended in incubation medium and cultivated at 37° C. in a humidified atmosphere with 5% CO₂. The acinar tissue died rapidly (within two days) under these conditions and the dying differentiated cells were separated from the neighboring cells without damaging them (protective isolation), while the non-dying stem cells sank to the bottom, where they adhered. The differentiated acini cells are not capable of doing this. The incubation medium was changed for the first time on the second or third day after sowing, a large part of the freely floating acini and acinar cells being removed at this time. At this point in time, the first stem cells and/or their precursors had settled on the bottom and began to divide. The change of medium was then repeated on every third day and differentiated acinar pancreatic cells were removed at each change of medium.

On the seventh day in culture the cells were passaged with a solution consisting of 2 ml PBS, 1 ml trypsin (+0.05% EDTA) and 2 ml incubation medium. The cells separated from the bottom of the culture dish. The cell suspension was centrifuged for 5 minutes at approximately 1000 rpm (Beckmann GPR centrifuge), the supernatant was removed by suction and the cells were re-suspended in 2 ml incubation medium, transferred to a medium cell culture flask and 10 ml incubation medium were added.

On the fourteenth day in culture, the cells were passaged again, but this time with 6 ml PBS, 3 ml trypsin/EDTA and 6 ml incubation medium. The cell suspension is centrifuged for 5 minutes at 1000 rpm, the supernatant was removed by suction and the cells were re-suspended in 6 ml incubation medium, transferred to 3 medium cell culture flasks and 10 ml incubation medium each were added.

On day 17, a third passaging was performed on a total of 6 medium cell culture flasks, and on day 24, a fourth passaging was performed on a total of 12 medium cell culture flasks. At the latest now, all primary cells except for the stem cells were removed from the cell culture.

The stem cells can be further cultivated and passaged and sown as often as desired. The sowing is preferably performed in a density of 2×10⁵ to 4×10⁵ cells/cm² in incubation medium.

### EXAMPLE 3

The isolation and cultivation from exocrine tissue of the parotid gland of a human being were performed by analogy with the pancreas protocol with the following deviations:

1. The exocrine tissue of the parotid gland was a mixture of acinar tissue and tubular tissue.

2. Since salivary glands contain less proteases and amylases than the pancreas, it is possible to store the salivary gland tissue for a while in the refrigerator at approximately 4° C. before the workup without damaging the tissue too much. In the concrete example, the storage time was 15 hours and entailed no negative consequences for the isolation of the desired stem cells.

The following example 4 describes in detail a working protocol for producing organoid bodies.

### EXAMPLE 4

The undifferentiated cells are trypsinated off with a solution of 10 ml PBS, 4 ml trypsin, 8 ml differentiation medium and are then centrifuged off for 5 minutes. The resulting pellet is re-suspended in differentiation medium in such a manner that a dilution of 3000 cells per 100 μl medium is adjusted. The cells are subsequently well-suspended again with a 3 ml pipette.

The cover is removed from bacteriological Petri dishes that had been previously coated with 15 ml PBS (37° C.) per plate and the cover is turned over. Approximately fifty 20 μl drops are put on a cover using an automatic pipette. The cover is then rapidly turned over and placed on the Petri dish that is filled with differentiation medium so that the drops hang downward. The Petri dishes are subsequently carefully placed in the incubator and incubated for 48 hours.

After this, the aggregated cells forming the organoid bodies in the hanging drops are transferred from four covers at a time into a bacteriological Petri dish with 5 ml incubation medium with 20% FCS and cultivated for 96 hours further.

The organoid bodies are now carefully collected with a pipette and transferred into cell culture vessels with differentiation medium and coated with 0.1% gelatin. In an especially preferred embodiment of the process, 6 cm Petri dishes coated with 0.1% gelatin are used as culture vessel into which 4 ml differentiation medium was placed before and which were subsequently charged with 6 organoid bodies each. Chamber slides coated with 0.1% gelatin constitute another preferred culture vessel, into which 3 ml differentiation media was placed and that were subsequently charged with 3 to 8 organoid bodies each. In addition, 24-well microtiter plates that were coated with 0.1% gelatin and into which 1.5 ml per well differentiation medium were placed and that were subsequently charged with 4 organoid bodies each can also be used.

When cultivated in this manner, the ability of the cells to differentiate into the organoid body is activated and the cells can differentiate in particular into hormone-producing cells. The cells can be stored and cultivated as organoid bodies as well as individual cells and retain their pluripotency.

(II) Differentiation Into the Organoid Bodies and/or Tissue Bodies

After the isolation and optional aggregation, the stem cells or, optionally, already spontaneously differentiated hormone-producing cells are present in the particular cell cultures or organoid bodies. In the further process the stimulated differentiation of the stem cells or the stimulated growth of the already differentiated cells is performed according to step 200 (FIG. 1). In this purpose, one or more of the above-mentioned stimulation treatments is/are carried out. The stimulation of the differentiation can be performed with the above-mentioned differentiation medium (I) in such a way that in general a differentiation of the stem cells or organoid bodies occurs. The inventors observed that the differentiation yields, in addition to other cell types, in particular the hormone-producing cells, that are then selected in the further processing (step 300 in FIG. 1). Alternatively, the differentiation is provided with the above-mentioned other differentiation media, with which predominantly a differentiation into the hormone-producing cells occurs and by the use of which the following steps of identification and selection are simplified. Following Example 5 refers to the stimulation with the differentiation medium (I) whereas the remaining examples refer to the other differentiation media and stimulation techniques.

### EXAMPLE 5

For the induction of the differentiation, stem cells after the 42^{nd} day of cultivation were preferably used. The use of stem cells after the 3^{rd} or 4^{th} passage or of cells that had been stored for 12 to 18 months at the temperature of liquid nitrogen was also possible without problems.

At first, the cells were transferred in differentiation medium (I) with the above-mentioned composition and adjusted to a density of approximately 3×10⁴ cells/ml, for instance, by the trypsin treatment of a stem cell culture in culture medium, 5 minute centrifugation at 1000 rpm and re-suspension of the pellets in differentiation medium (I) and dilution to the extent required.

Subsequently, approximately 50 20 μl drops (600 cells/20 μl) were placed on the inside of the cover of a bacteriological Petri dish (packed tips) and the covers carefully placed over the Petri dishes filled with PBS so that the drops hang downward. A new tip was used for each cover. The Petri dishes were subsequently carefully set in the incubator and incubated for 48 hours at 37° C.

After this, the cells aggregated in the hanging drops, the organoid bodies, were transferred from each four covers into one bacteriological Petri dish with 5 ml incubation medium with 20% FCS (hold cover obliquely and rinse the organoid bodies off with approximately 2.5 ml culture medium) and cultivated for another 5 to 9 days, preferably for 96 hours.

The organoid bodies were now carefully collected with a pipette and transferred into cell culture vessels coated with 0.1% gelatin and containing differentiation medium (I). The organoid bodies then propagated and grew partially in individual cell colonies that could be further individualized, propagated and individualized again. In an especially preferred embodiment of the process, 6 cm Petri dishes coated with 0.1% gelatin were used as culture vessel, into which 4 ml differentiation medium (I) had been placed and which were charged with 6 organoid bodies each. Another preferred culture vessel was represented by chamber slides coated with 0.1% gelatin, into which 3 ml differentiation medium had been placed and which were subsequently charged with 3 to 8 organoid bodies each and Thermanox plates (Nalge Nonc International, USA) for studies with an electronic microscope. Another alternative were 24-well microtiter plates that were coated with 0.1% gelatin, into which 1.5 ml per well differentiation medium (I) had been placed and that were each subsequently charged with 4 organoid bodies.

In a preferred embodiment of the process, organoid bodies were cultivated for approximately 7 weeks in the gelatin-coated 6 cm Petri dishes and thereafter individual organoid bodies were cut out with the Microdissector (Eppendorf, Hamburg, Germany) in accordance with the instructions of the manufacturer and then transferred, for instance, onto fresh 6 cm Petri dishes, chamber slides or Thermanox plates.

### EXAMPLE 6

The stimulation with the other differentiation media mentioned above is performed in an analogous manner.

### EXAMPLE 7

FIG. 6 illustrates the stimulation (step 200) of a stem cell 1 on a substrate 21 by molecular signal or differentiation factors 5 contained in the cultivation medium. Cell receptors 1 a are located on the surface of stem cell 1. When coupling the signal or differentiation factors on cell receptors 1a, stem cell 1 is imprinted and the differentiation to the desired hormone-producing cell starts. The molecular signal or differentiation factors 5 comprise known biological macromolecules, cellular components of pancreatic cells or, in particular, cells, cellular components or cell groups that are located in the endocrine tissue of the pancreas in the vicinity of the islets of Langerhans are used.

### EXAMPLE 8

FIG. 7 illustrates accordingly the imprinting of stem cell 1 with an already differentiated cell 6, on whose membrane surface appropriate signal or differentiation factors are fixed or are naturally present.

### EXAMPLE 9

According to another variant, a gene activation of the stem cells can be envisaged as is described, for instance, in DE 102 90 025 T1.

### EXAMPLE 10

FIG. 8 illustrates the insulin production of the differentiated cells with a microscopic image of insulin-producing cells that were obtained from the exocrine pancreas of a human being. Corresponding results have been found with salivary glands of a human being or of other vertebrates. The points marked with arrows represent the insulin produced by the cells. The white line corresponds to a length of 20 μm in the original.

The features of the invention disclosed in the above description, the claims and the drawings can be significant individually as well as in combination for the realization of the invention in its various embodiments.

1. A process for generating cells producing pancreatic hormone, comprising:
cultivation and differentiation of pluripotent stem cells obtained from differentiated exocrine glandular tissue of an organism, and having a capacity to form organoid bodies.

2. The process according to claim 1, wherein stem cells isolated primarily from the organism are cultivated and differentiated.

3. The process according to claim 1, wherein an aggregation of the stem cells to organoid bodies is provided.

4. The process according to claim 3, wherein the differentiation of the stem cells is carried out in the organoid bodies.

5. The process according to claim 3, wherein stem cells isolated secondarily from the organoid bodies are cultivated and differentiated.

6. The process according to claim 1, wherein a stimulation of the generation of cells producing pancreatic hormone that comprises a stimulated propagation of the cells producing pancreatic hormone and/or a stimulated differentiation of the stem cells is provided.

7. The process according to claim 6, wherein the stimulation comprises;
(a) at least one stimulation treatment selected from the group consisting of:
treatment with supernatants of a primary culture of endocrine pancreas,
treatment with supernatants of cell lines of Endocrine pancreas,
co-culture with differentiated cells of endocrine pancreas,
co-culture with cell lines of endocrine pancreas, and
treatment with immobilized molecular growth factors,
(b) activation of at least one gene involved in the differentiation of stem cells into the cells producing pancreatic hormone, and
(c) treatment with molecular growth factors dissolved in a liquid.

8. The process according to claim 7, wherein the treatment with immobilized molecular growth factors comprises a cellular imprinting with molecular differentiation factors immobilized on a carrier.

9. The process according to claim 8, wherein a synthetic substrate, a cell membrane or a three-dimensional matrix substrate is used as carrier.

10. The process according to claim 1, wherein an identification and selection of the cells producing pancreatic hormone is provided.

11. The process according to claim 10, wherein the selection of the cells producing pancreatic hormone comprises a cell sorting process.

12. The process according to claim 10, wherein non-identified and selected cells are subjected to a further cultivation and differentiation.

13. The process according to claim 1, wherein stem cells obtained from secretory glands or glands of a gastrointestinal tract of the organism are used.

14. The process according to claim 13, wherein stem cells obtained from a pancreas or a salivary gland of the organism are used.

15. The process according to claim 1, wherein stem cells from glandular tissue that is acinar tissue are used.

16. The process according to claim 1, wherein stem cells from a vertebrate are used.

17. The process according to claim 16, wherein stem cells from a primate are used.

18. The process according to claim 1, wherein the cells producing pancreatic hormone are used for pharmaceutical applications.

19. The process according to claim 18, wherein the cells producing pancreatic hormone are used for treating pancreatic diseases, a metabolic syndrome or metabolic diseases.

20. The process according to claim 19, wherein the cells producing pancreatic hormone are used for treating diabetes, hyperglycemia or impaired glucose tolerance.

21. The process according to claim 1, wherein the cells producing pancreatic hormone produce insulin.

22. An isolated cell producing pancreatic hormone, the cell having been generated from a pluripotent stem cell isolated from differentiated exocrine glandular tissue of an organism, and having a capacity to form organoid bodies.

23. The isolated cell producing pancreatic hormone according to claim 22, which is a human cell.

24. A cellular composition containing a plurality of cells producing pancreatic hormone according to claim 22.

25. The cellular composition according to claim 24, wherein the cells producing pancreatic hormone are generated by a process comprising cultivation and differentiation of pluripotent stem cells obtained from differentiated exocrine glandular tissue of an organism, and having a capacity to form organoid bodies.

26. The cellular composition according to claim 24, which additionally contains other cell types.

27. The cellular composition according to claim 26, wherein the other cell types comprise stem cells and/or neighboring cells of islets of Langerhans in pancreatic tissue.

28. The cellular composition according to one of claims 24, which contains a casing or matrix material.

29-31. (canceled)

32. Artificial islets of Langerhans containing a cell according to claim 22.

33. A pharmaceutical composition containing:
at least one cell according to claim 22, and
a pharmaceutical carrier substance.

34. The pharmaceutical composition of claim 33, which is autologous.

35. A pharmaceutical composition containing:
at least one cellular composition according to claim 24, and
a pharmaceutical carrier substance.

36. The pharmaceutical composition of claim 35, which is autologous.

37. Artificial islets of Langerhans containing at least one cellular composition according to claim 24.

38. A medical transplant or medical device comprising at least one cell according to claim 22.

39. A medical transplant or medical device comprising at least one cellular composition according to claim 24.

40. The process according to claim 1, wherein the stem cells are from a mammal.

41. The process according to claim 1, wherein the stem cells are from a human.
